(19) United States
(12) Patent Application Publication
(10) Pub. No.: US 2007/0270512 A1
(43) Pub. Date: Nov. 22, 2007

(54) Methane conversion to methanol

(76) Inventor: John Lee Edwards, (US)

Correspondence Address: John L. Edwards, 3953 Hamilton Club Circle, Sarasota, FL, 34242, US

(21) Appl. No.: 11/803,511

(22) Filed: May 15, 2007

Related U.S. Application Data
(60) Provisional application No. 60/801,175, filed on May 17, 2006.

(51) Int. Cl. C07C 27/06

(52) U.S. Cl. 518/702

(57) ABSTRACT

Disclosed are a method and apparatus with alternate embodiments that use commercial chemical free radical initiators to directly convert methane to methanol in a low energy, one step, catalytic reaction. In one embodiment an apparatus converts “packets” of methane gas to liquid methanol. Methane gas and aqueous hydrogen peroxide are injected into the packet apparatus through valved ports and mixed in a structurally robust, rotating reaction chamber coated with metal cation catalyst. Although operating at low temperature and pressure, the rotating chamber is sufficiently strong enough to contain spontaneous ignition of the mixture should that occur. The reactive oxygen species, primarily hydroxyl radicals, released in the chamber of mildly elevated temperature, oxidize the methane molecule, replacing one hydrogen atom on its molecule with one stable hydroxyl OH molecule, converting vapor methane CH₄ to vapor methanol CH₃OH. Vapor exiting the exhaust port condenses on ambient temperature drip screens to stable liquid methanol. An alternate continuous process embodiment uses flow-through conversion screens coated with immobilized dry chemicals. Water vapor entering the apparatus releases molecular oxygen from screens coated with dry urea hydrogen peroxide. A circulating endless belt screen is used to continual recoat the dry urea hydrogen peroxide. Reactive oxygen species are generated on successive conversion screens through which the molecular oxygen, and methane gas entering the apparatus, must pass. Metal cation catalyst molecules physically entrapped within interstitial cavities of a three dimensional, fixed porous colloidal matrix coating on the screens create powerful hydroxyl radicals from the molecular oxygen. These reactive species in turn convert methane molecules impacting the conversion screens to methanol molecules. The method and apparata operate at modest temperature, pressure, and energy requirement levels, and are scalable from portable units to industrial scale methanol refineries.

This application claims priority from provisional filing 60/801,175 “Methane Reforming” filed May 17, 2006.

### BACKGROUND OF THE INVENTION

This invention relates broadly to the conversion of gaseous methane to fuel grade liquid methanol. More specifically, the invention is a novel method and apparatus for catalytically converting methane to methanol at ambient temperature and pressure, with very low energy input, using commercially available specialty chemicals.

The world need for alternative energy is well known. Methane-containing natural gas is a clean and effective alternative energy resource. But it is difficult and costly to transfer.

Natural gas has such a low density that it is uneconomical to bring to market in gas form except through an accessible pipeline system (which is uneconomical beyond 3,000 kilometers). Its principal component, Methane, has a −164 degree C. boiling point, necessitating expensive compression and liquid nitrogen refrigeration for liquid transport. This is why Liquefied Natural Gas (LNG) requires such a huge investment for liquefaction, refrigerated transport, and receiving terminals. Despite the costs, part of the U.S. alternative energy initiative includes major investments to increase the current five LNG receiving terminals at major U.S. seaports to 55.

Huge deposits of methane worldwide are economically “stranded” because of the high capital costs of using existing technology to make it transportable. A system, based on new technology, that would economically convert methane into fuel that could be transported and stored economically, would provide for the first time the capability to monetize the huge stranded gas reserves worldwide, and ultimately tap huge ocean floor deposits of methane hydrate reserves to supply world energy needs for 200 years after petroleum reserves have been exhausted.

Methane is the simplest organic molecule in nature consisting of one carbon atom bonded with four hydrogen atoms. Methanol is only one atom change away, with one of the hydrogen atoms replaced by an OH hydroxyl. Methane is the simplest of the alkenes, with a chemical formula of CH₄. Methanol, the initial product of methane oxidation, is a desirable product of conversion because it retains much of the original energy of methane in a room temperature transportable, storable liquid that can be used directly as a fuel or converted to other valuable products. In addition to its commercial importance as a chemical feedstock, there are many advantages of using methanol alcohol for broad use as an environmentally compatible and efficient vehicle fuel. It has lower combustion emissions of greenhouse gasses, evaporative hydrocarbons, and particulate pollutants, yet has a high energy content. Methanol can even be converted into boiling grade gasoline if necessary.

Existing technologies for converting methane to methanol include destruction of methane to form a synthesis gas (a mixture of carbon monoxide and hydrogen), followed by indirect liquefaction steps. In these and other processes of producing methanol it is necessary to provide production facilities where a large amount of hydrogen and carbon monoxide are produced by environmentally dangerous processes, by methane cracking and coking. These facilities are environmentally limited to certain heavy industrial areas of the world. Attempts have been made to carry out catalytic conversion of the syn gas to methanol, but these energy intensive processes typically have poor conversion efficiencies, slow reaction rates, and are not economically competitive.

Good environmental alternatives generally still necessitate large capital costs, and high energy requirements. U.S. Pat. No. 4,374,288 to Scragg discloses combining methane and oxygen in a high energy electromagnetic field strong enough to atomize the oxygen for combining with methane.

Direct conversion processes have suffered from extreme operating requirements and low methane conversion and poor selectivity to methanol. Basic research has shown that gas-phase transition metal oxide cations can convert methane to methanol, with efficiency and selectivity dependent upon each metal's theoretical thermodynamics, and experiment pressure and collision energy.¹ ¹ Metz, Ricardo, Methane-to-Methanol Conversion by Gas-Phase Transition Metal Oxide Cations: Experiment and Theory, Department of Chemistry, University of Massachusetts, Amherst, Mass., August 2002.

United Kingdom Pat. No. 1,244,001 discloses oxidation of methane over a (Mo₂O₃)Fe₂O₃ catalyst on silica/aluminum at high temperature and pressure. U.S. Pat. No. 5,220,080 to Lyons et al discloses catalytic oxidation of methane employing surface oxide chromate catalyst on a metal oxide support of silica, alumina, magnesia, titania, or zirconia. U.S. Pat. No. 5,504,262 to Vanderspurt et al discloses methods for making mixtures of ethanol and methanol in a slurry reactor by reacting methane, water, and an iron-based acidic solution with platinum or palladium.

Numerous U.S. patents have been issued for photocatalytic conversion of methane to methanol. Those that require UV light impose special health and equipment factors. U.S. Pat. No. 6,156,211 to Gonzolez-Martin et al discloses the use of porous semiconductor membranes to photochemically convert methane in the presence of UV illumination. U.S. Pat. No. 6,156,279 to Sherman discloses the use of UV lamp illuminated semi permeable membrane catalysts in continuously flowing water to create hydroxyl radicals to cleave the carbon-hydrogen bonds of methane.

Visible light systems have generally suffered from inadequate concentration of free radicals and therefore slow conversion efficiencies and slow rates. U.S. Pat. No. 5,720,858 to Noceti et al uses visible light illuminating metal doped transition metal oxide semiconductor catalysts to convert 5.5% methane to methanol in a slow reaction rate process. U.S. Pat. No. 6,267,849 to Taylor et al discloses converting methane stored in natural gas hydrates to methanol and hydrogen through exposure of a typical, metal doped oxide salt, radical initiator, photo catalyst to UV or visible light.

### BRIEF SUMMARY OF THE INVENTION

The present invention uses reactive oxygen species produced through a catalytic reaction to convert methane to methanol.

The invention uses vapor phase aqueous chemical compounds combined in rotational valved chambers with reactive oxygen species to alter the methane molecule, and control any potential spontaneous combustion during the conversion process.

Alternatively, the invention uses immobilized dry chemical compounds bound to the surface of porous screens to generate the reactive oxygen species that alter the methane molecule.

The energy requirements of this novel approach are modest compared to conventional methane reforming technology, and compared to the energy necessary for photocatalytic generation of reactive oxygen species.

More particularly, this invention impacts methane molecules with reactive oxygen species in valved chambers or on three dimensional reactive surfaces, where such species chemically convert the methane into fuel grade liquid methanol.

Accordingly, it is a primary object of the present invention to provide a new and improved method and apparatus for converting methane to methanol thereby.

It is a further object of this invention to provide a method and apparatus for producing methanol by environmentally acceptable techniques.

It is a further object of this invention to provide several apparata embodiments for carrying out methods that achieve the foregoing objects and which are relatively simple in construction, effective in production, and economical to operate.

It is a further object of this invention to provide such methods and apparata that can easily be used in either a portable configuration to produce small quantities of methanol at remote locations, or to produce large quantities of methanol in an industrial scale methane-methanol refinery.

These and other objects will be made manifest when considering the following detailed specification when taken in conjunction with the appended drawing figures.

### BRIEF DESCRIPTION OF THE DRAWINGS

FIG. 1 is a drawing of an embodiment of the invention comprising a rotating cell reactor which receives injected methane and injected hydrogen peroxide diluted in water vapor, exhausts vaporized methanol, and condenses the exhaust to stable liquid methanol.

FIG. 2 is a drawing showing the reactor of FIG. 1 being physically driven by a methane-powered Wankel rotating motor whose drive shaft is a common axle.

FIG. 3 is a drawing of an alternate continuous flow embodiment of the invention comprising a closed loop duct containing a fan, oxygen release screens, catalytic conversion cartridges, methane and water vapor inlets, a methanol outlet, and a waste gas outlet.

FIG. 4 is a drawing showing the use of an endless belt screen in a continuous methane conversion process to continually replenish dry chemicals on the oxygen release screen of FIG. 3.

FIG. 5 is a drawing of a catalytic conversion cartridge of FIG. 3 using metal cation catalyst embedded on the surface of the non-woven mesh material.

FIG. 6 is a drawing showing a cutaway of the multiple screens of the FIG. 5 conversion cartridge.

### DETAILED DESCRIPTION OF THE INVENTION

The present invention provides a direct, one-step chemical conversion method and apparatus wherein methane molecules and hydrogen peroxide molecules diluted in water vapor are chemically converted into pure, fuel grade liquid methanol.

The conversion reaction uses catalyst agents to create reactive oxygen species, primarily hydroxyl radicals, from hydrogen peroxide diluted in water vapor, which oxidize the methane molecule to a methanol molecule in a one-step chemical conversion

Hydrogen peroxide (H₂O₂) is a very pale blue liquid which appears colorless in a dilute solution, slightly more viscous than water. It is a weak acid.

Hydrogen peroxide is manufactured today almost exclusively by the autoxidation of 2-ethyl-9,10-dihydroxyanthracene to 2-ethylanthraquinone and hydrogen peroxide using oxygen from the air.⁴ The anthraquinone derivative is then extracted out and reduced back to the dihydroxy compound using hydrogen gas in the presence of a metal catalyst. Process economics depend on recycling effectiveness of the solvents and the catalyst. The overall equation for the process is:

H₂+O₂→H₂O₂

⁴ Wikipedia, Manufacturing Process, Hydrogen Peroxide.

Although hydrogen peroxide is manufactured by a process that consumes energy and/or other chemical resources, it is a relatively low cost commodity to convert vast energy reserves of stranded methane to a valuable, transportable, fuel alternative to petroleum. In 1994, world production of hydrogen peroxide was around 1.9 million tons, most of which was at a concentration of 70% or less. In that year bulk 30% product sold for around US $0.54 per kg, equivalent to US $0.68 per lb on a “100% basis”.⁵ ⁵ Ibid.

Today, the price of 50% diluted hydrogen peroxide to pulp and bleaching plants is about $550 per ton which is $1.25 per gallon. If a gallon of 50% hydrogen peroxide can make 10 gallons of methanol, the raw material cost would be 12.5 cents per gallon. Furthermore, more than half the variable cost of hydrogen peroxide production is for hydrogen. The hydrogen can be produced from electrolysis of water at hydroelectric plants where electric power is cheap. But the hydrogen can also be produced from free methane, theoretically also made at the remote site of methane conversion to methanol.⁶ ⁶ Hydrogen Peroxide and Sugar, American Energy Independence, http://www.americanenergyindependence.com/peroxide.html, 2007.

Hydrogen peroxide has strong oxidizing properties and is therefore a powerful bleaching agent that has found use as a disinfectant, as an oxidizer, and in rocket propulsion. It is one of the most powerful oxidizers known—stronger than chlorine, chlorine dioxide, and potassium permanganate:

| Oxidant | Oxidation potential, V |
|---|---|
| Fluorine | 3.0 |
| Hydroxyl radical | 2.8 |
| Ozone | 2.1 |
| Hydrogen peroxide | 1.8 |
| Potassium permanganate | 1.7 |
| Chlorine dioxide | 1.5 |
| Chlorine | 1.4 |

There are many reactions where hydrogen peroxide acts as a reducing agent, releasing oxygen as a by-product. It always decomposes exothermically into water and oxygen gas spontaneously, at a rate dependent on temperature and peroxide concentration.

Because oxygen is formed during the natural decomposition, there is a resulting increase in pressure of any container hosting the reaction. Peroxide vapor can react with alcohols and hydrocarbons to form contact explosives, and the vapor itself can detonate above 70 C. Hydrogen peroxide is Generally Recognized As Safe (GRAS) as an antimicrobial agent, an oxidizing agent and more by the US Food and Drug Administration. Hydrogen peroxide is used as a toothpaste when mixed with baking soda and salt, and is also used in the treatment of acne.

Through catalysis, hydrogen peroxide can be converted into hydroxyl radicals which have a reactivity second only to fluorine.

It is known in the art that a hydroxyl radical is produced by the reaction of hydrogen peroxide and a metal cation. This hydroxyl radical will react with a methane molecule to produce a methyl radical and water in the following reaction:

CH₄+OH′→CH₃′+H₂O

The methyl radical CH₃′ then reacts with a water molecule present to produce methanol and hydrogen:

CH₃′+H₂O→CH₃OH+½H₂

In the end product the OH′ hydroxyl radical has replaced one hydrogen atom on the methane molecule with one stable hydroxyl OH molecule to create methanol. Both liquid methanol and hydrogen gas are commercially valuable as fuels or chemical intermediates.

The combined reactions are mildly endothermic and require a temperature elevation of about 8 C to 10 C. This is a minor energy input requirement of the process which is available from frictional heat and compression heat of the conversion apparatus.

Various initiator-catalyst combinations can be used, including the metal cation copper sulfate CuSO₄, and transition metal oxide cations PtO+, FeO+, and MnO+. Copper sulfate is a common salt of copper. It exists in nature as a series of compounds that differ in their degree of hydration. The most common form is copper sulfate pentahydrate (CuSO₄.5H2O), (the mineral called chalcanthite). Copper sulfate decomposes before melting. The pentahydrate form dehydrates, turning from blue to white as it loses four water molecules at 110° C. and all five at 150° C. Returned to a lower temperature it will rehydrate the evaporated water turning back to a blue color. (It is toxic to aquatic organisms, and in man for inhalation, ingestion, or prolonged skin exposure.)

Another reactive mechanism that can be employed with this method is the use of methane monooxygenase enzymes in membrane bound form, (pMMO). The pMMO, which is also primarily copper cation based, is used by bacteria (methanotrophs) to oxidize methane.

Commercially available forms of hydrogen peroxide can be used. Diluted hydrogen peroxide is commercially available diluted in purified water in concentrations from 3% as peroxide in drug stores for medically cleaning wounds to 95% concentration as a propellant. Hydrogen peroxide is also available commercially in dry chemical form as Dry Urea Hydrogen Peroxide. This dissolves in water to release molecular oxygen, from which a hydroxyl radical OH′ can be generated with a metal cation acting as an initiator-catalyst.

We have demonstrated production of liquid methanol from vapor phase methane using hydroxyl radicals generated by combining dry urea hydrogen peroxide CO(NH₂)₂.H₂O₂ (CAS# 124-43-6) with the metal cation copper sulfate CuSO₄ (CAS# 7758-99-8). The overall chemical reaction employed was:

H₂O₂+CH₄------->CH₃OH+H₂O (with CuSO₄ functioning as a catalyst)

The presence of liquid methanol product was confirmed by potassium dichromate color change.

The preferred pathway of the reaction was:

CO(NH₂)₂.H₂O₂+CuSO₄------->OH′+[waste]

CH₄+OH′→CH₃′+H₂O

CH₃′+H₂O→CH₃OH+½H₂

The Wankel motor is a rotating internal combustion engine that creates three independent sealed moving chambers of gas by housing a roughly triangular shaped rotor with three equally wide faces in an oval-like epitrochoid shaped housing. (An epitrochoid is formed when a circle rolls around another circle and a point on the outer circle transcribes a path.) The rotor both rotates around an offset crank and makes orbital revolutions around the central shaft. The output shaft is geared to provide torque via the complex planetary motion. Apexes of the rotor act as a seal.⁷ ⁷ Pruitt, Jeremy, Theory and Operation of Wankel-rotary Engines. Stephan F. Austin State University, Physics Department, October 2003.

Wankel rotary engines have valving with few moving parts and are low compression compared to reciprocating engines. Their design compression ratio is adjustable by configuration of the rotor surface shape and displacement pockets.

The Wankel configuration is ideal to use as a rugged mixing platform for the conversion of packets of methane gas to methanol. It is a valved device with few moving parts, can be built from non-metal castings to operate at low friction and low temperature, and can be adapted as a very low compression ratio mixing device capable of withstanding unintended spontaneous combustion of the feed gas mixture.

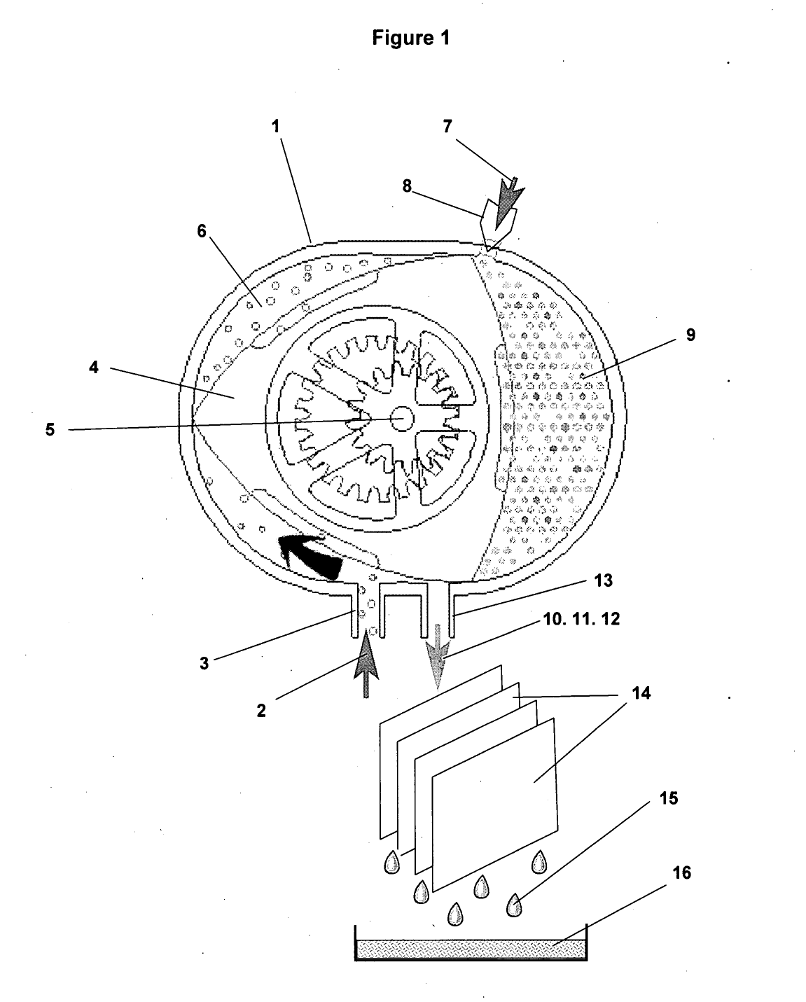

FIG. 1 is a drawing showing one embodiment of the invention comprising a Wankel-like rotating cell reactor 1 which converts methane gas 2 to liquid methanol 16. Methane gas is injected into the inlet port 3 during the intake cycle. The packet volume of feed gas is isolated in section 6 of the housing by planetary rotation of rotor 4 around geared drive shaft 5. Section 9 of the housing is coated with deposited metal cation catalyst such as copper sulfate. Hydrogen peroxide diluted water vapor is injected into the volume packet via inlet port 8 as the rotor 4 moves the volume to section 9 of the chamber, In section 9 the methane gas is converted to methanol vapor 10, hydrogen gas 11, and water vapor 12 which is exhausted via outlet port 13. Methanol vapor 10 is condensed on drip plates 14 to stable liquid methanol 15 and collected at station 16.

Essentially, hydroxyl radicals are produced in the rotating chamber by the reaction of molecular oxygen released from hydrogen peroxide, and the coated metal cation catalyst. These hydroxyl radicals react with methane molecules to produce a methyl radical and water. The methyl radical then reacts with an additional water molecule to produce methanol and hydrogen, both of which are commercially desirable as fuels or chemical intermediates. In the end product the hydroxyl radical has replaced one hydrogen atom on the methane molecule with one stable hydroxyl OH molecule to create methanol.

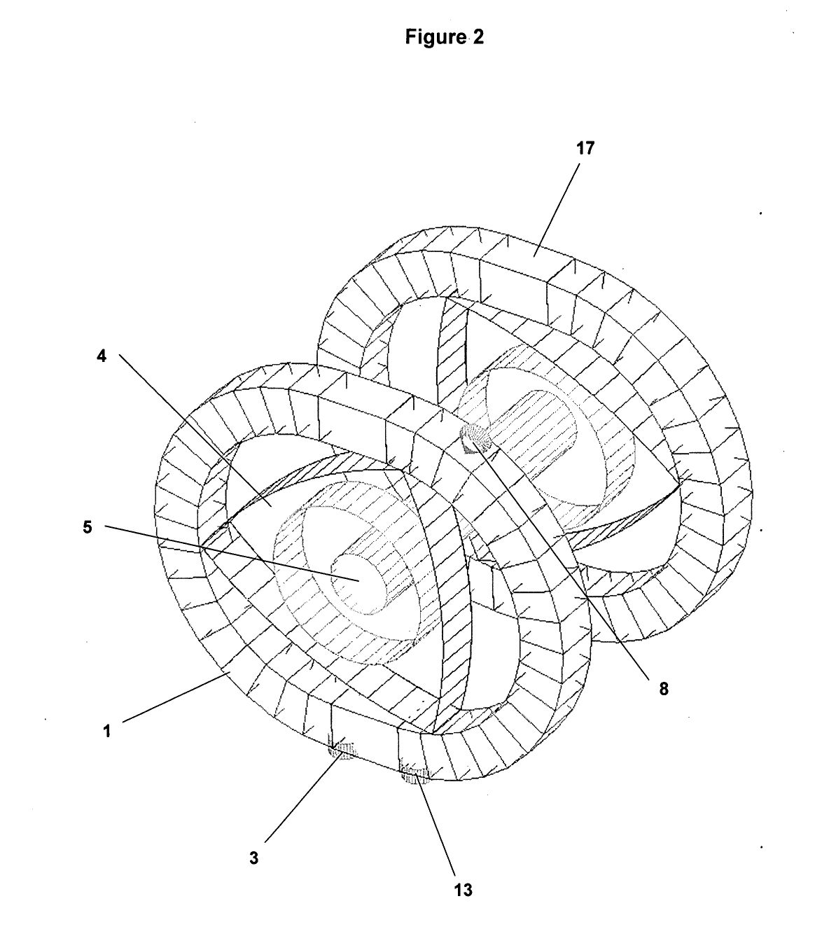

FIG. 2 shows rotating cell reactor 1 being physically driven by a methane-powered Wankel rotating motor 17 whose drive shaft 5 is a common axle with reactor 1. The motor driving reactor 1 may be virtually any other rotating motor driven by electricity or fuel, and it may be connected to the reactor 1 axle via clutches and transmission mechanisms as well. Such connections generate additional frictional energy losses and higher capital-operating-maintenance-repair costs. The embodiment shown in FIG. 2 is elegantly simple, with low maintenance; it can run unattended for long periods of time at remote sites. Both the reactor 1 and the Wankel motor 17 can use a common source of methane feed gas for methanol conversion and as the energy supply to rotate the reactor.

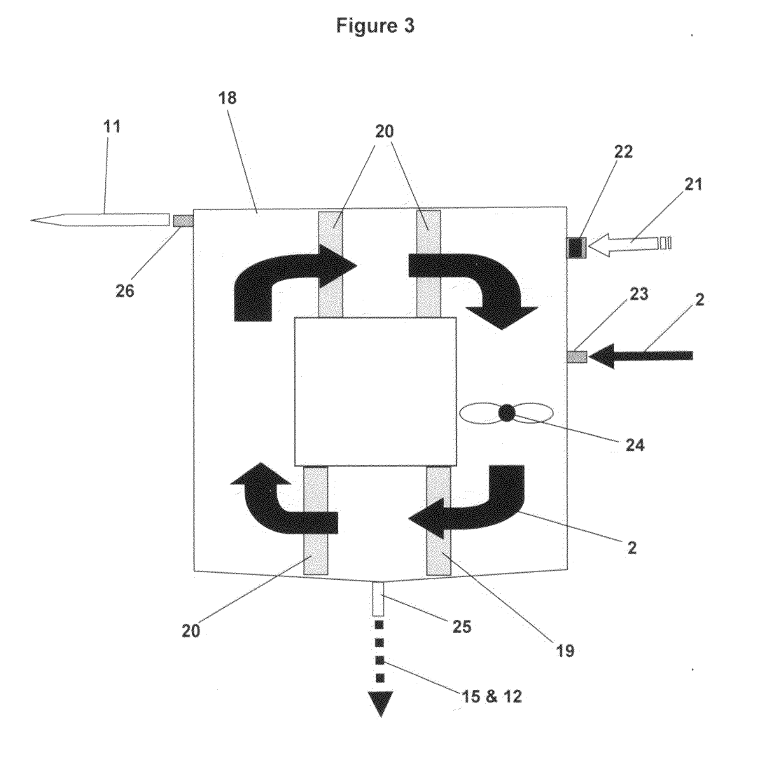

FIG. 3 is a drawing showing an alternate embodiment of the invention for a continuous flow process. Reactor 18 comprises a closed loop duct containing a fan 24, oxygen release screen 19, catalytic conversion cartridges 20, water vapor 21 into inlet port 22, and methane 2 into inlet port 23. Methane 2 and water vapor 21 entering apparatus 18 are circulated through the oxygen release screen 19 and catalytic conversion cartridges 20 by fan 24. The moving water vapor impacts the oxygen release screen 19 and the dry chemical coating the screen's surface, releasing molecular oxygen into the flowing gas stream. The molecular oxygen passes through downstream catalytic conversion cartridges 20. Hydroxyl radicals are generated by the catalytic metal cations on the conversion cartridge screen 27 from the molecular oxygen impacting the screen surface. The hydroxyl radicals convert the methane molecules to methanol molecules plus hydrogen gas and more water vapor. The overall temperature of reactor 18 is controlled by exterior cooling and heat removal of the gas mixtures moving rapidly through the apparatus. Liquid methanol 10 and water vapor 12 drop to the bottom of the reactor 18 housing and out the exit port 25. Residual unconverted methane 2 and hydrogen gas 11 exit the apparatus at exit port 26.

Flowing water vapor 21 impacts the dry chemical coated surface on the fibers of oxygen release screen 19. The embodiment of the invention shown in FIG. 3 uses humectant coated screens to support the dry chemical coating that releases molecular oxygen. These humectant coated screens attract and bind incoming water vapor molecules for activation of the oxygen-containing dry chemicals also coating them. The humectant that is assuring the presence of water vapor at the reaction site can be biodiesel, glycerin, propylene glycol or other known and suitable humectants. A suitable source of molecular oxygen is dry urea hydrogen peroxide.

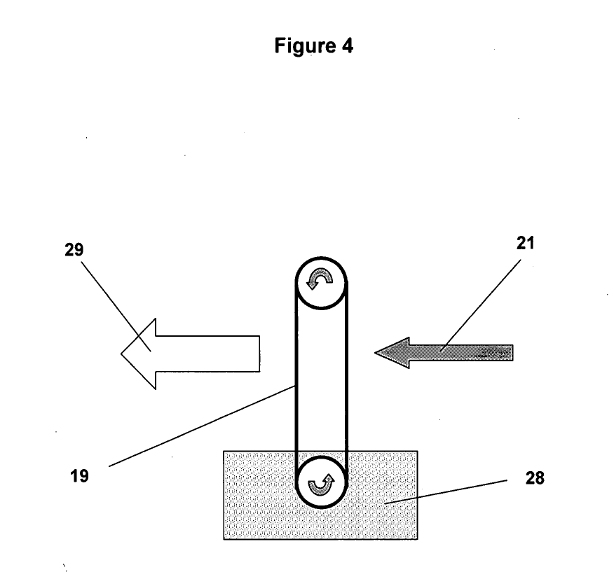

FIG. 4 is a drawing showing detail of the oxygen release screen configured as an endless belt for continuous replenishment of dry chemicals in a production flow process. Water vapor 21 passing through the moving screen 19 releases molecular oxygen 29. The endless screen 19 is continually recoated with dry chemical from supply station 28.

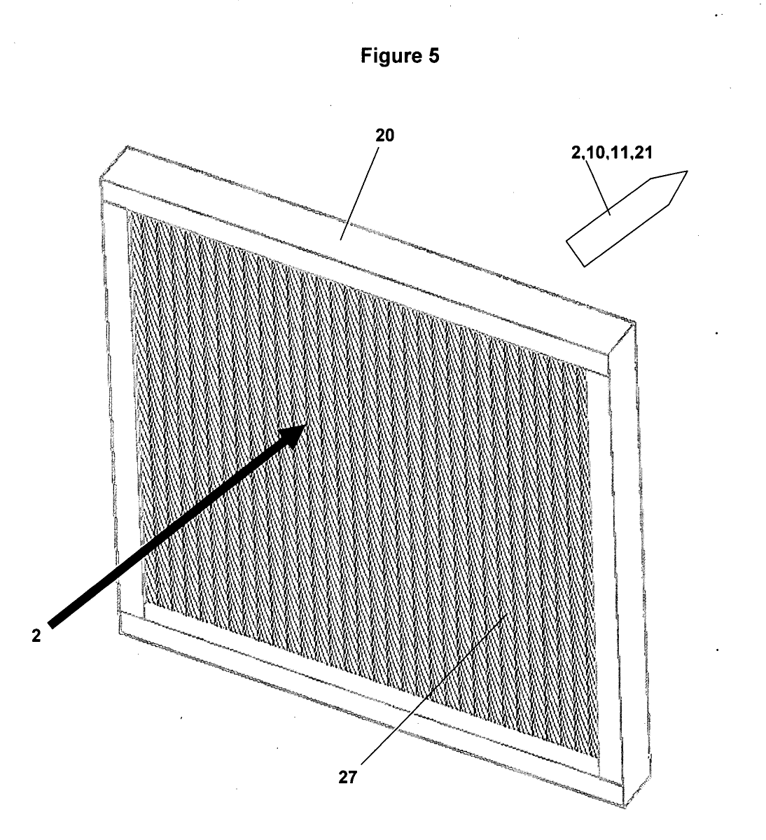

FIG. 5 is a drawing showing the detail of catalytic conversion cartridge 20 of the FIG. 3 embodiment using non-woven fibrous screens. The fibrous screen material 27 is coated with a sol gel colloidal suspension that sets up as a fixed, porous matrix. This sol gel coating is embedded with the metal cation catalyst, such as copper sulfate CuSO₄.

The sol gel coating on the conversion screen presents metal cation catalyst entrapped in a three dimensional porous matrix, with a large exposed surface area for the creation of hydroxyl radicals. When the sol gel coating on conversion screen fibers 27 is properly compounded and the proper number of coats are applied and dried, it provides a stable, high surface area, three dimensional dry carrier structure for molecular oxygen to react with embedded metal cation initiators. The catalysts are physically entrapped in the interstitial cavities of the three dimension matrix network. This matrix permits molecular oxygen molecules, (and methane molecules) to move freely in and out of the porous matrix to react with the entrapped catalyst, providing a stable “breathable” surface covered with short-life hydroxyl radicals whose surface area is hundreds of times greater than that of using only the fiber's natural two dimensional curved surface alone.

Methane molecules circulating in reactor 18 of FIG. 3 under the influence of fan 24, pass through catalytic conversion cartridges 20. Many of the suspended molecules of the moving methane gas will impact the hydroxyl radical covered surface of the mesh comprising conversion screen 27 and be converted to methanol vapor.

In FIG. 5 residual unconverted methane 2, plus methanol gas 10, hydrogen gas 11, and water vapor 21 continue on to the next conversion cartridge station. Liquid methanol 15 and liquid water 12 drop to the bottom of the cartridge housing under the influence of gravity and out the exit port 25.

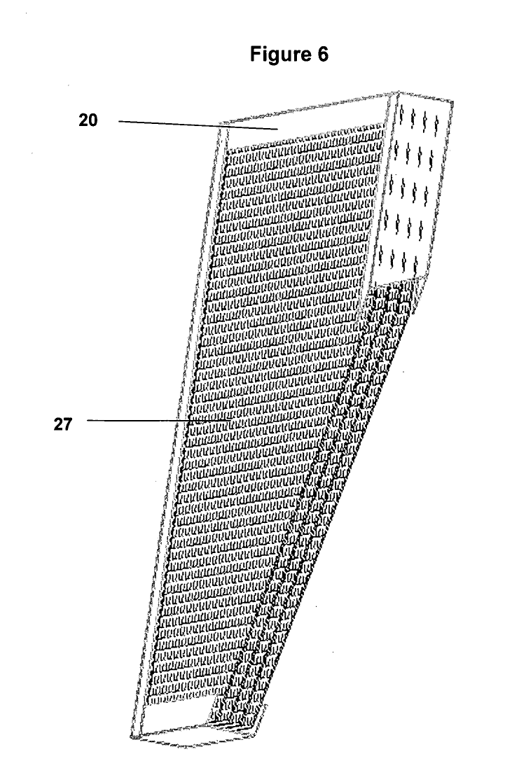

Application to Industrial Scale Methanol Refinery The invention can be in the form of an array of large dimension conversion cartridges, for installation into either portable apparatus, or into ducts feeding large scale methanol refinery systems. FIG. 6 is a drawing showing the cutaway of a multiple screen conversion cartridge of this invention installed in an industrial methane-to-methanol refinery process flow, each cartridge comprised of one or more metal cation bound conversion screens. Methane gas is converted on each screen 27 of a conversion cartridge 20 into methanol molecules, and in turn to fuel grade methanol liquid.

What is claimed is:

1. A method for converting methane to methanol, said method comprising the steps of
a. combining in a closed reaction chamber gaseous methane with water vapor carrying hydrogen peroxide;
b. providing a metal cation chemical in said reaction chamber to catalyze the chemical reaction;
c. elevating the temperature of said reaction chamber slightly to facilitate the chemical reaction;
d. the reactive oxygen species provided by the hydrogen peroxide replaces a hydrogen atom on the methane molecule with a hydroxyl molecule, converting vaporized methane molecules to vaporized methanol molecules;
e. the methanol vapor is condensed to stable liquid methanol.

2. A methane-to-methanol conversion apparatus comprising:
a. A reactor having a rotating mixing cell with valving properties;
b. Injecting methane and water vapor carrying hydrogen peroxide into the reactor's mixing cell during each rotation cycle;
c. Coating the interior surfaces of the mixing cell chamber with a metal cation catalyst;
d. Elevating the mixing cell temperature slightly by the rotation friction of the apparatus;
e. Converting in the mixing cell the methane vapor into methanol vapor and exporting such methanol vapor from the mixing cell later in each rotation cycle;
f. Condensation of methanol vapor to liquid methanol outside the mixing cell, such methanol liquid being stable at ambient temperature and pressure.

3. An alternate methane-to-methanol conversion apparatus comprising:
a. A closed loop duct where incoming feed gases are mixed and circulated by a fan;
b. An inlet port for methane, and an inlet port for water vapor;
c. An oxygen release screen interposed into said flow duct;
d. One or more catalytic conversion cartridges interposed into said flow duct;
e. Such conversion cartridge having an outer frame housing one or more internal conversion screens;
f. Said oxygen release screens and conversion screen materials fabricated of woven or nonwoven fibrous webs, or expanded mesh:
g. Such oxygen release screens and catalytic conversion screens having three dimensional porous colloidal dry chemical matrix coatings applied to the outer surface of their filter screen material in order to trap and present humectants and metal cation catalysts necessary for the process reactions;

4. The method of claim 1, wherein the hydrogen peroxide released from the water vapor is a source of molecular oxygen to create active oxygen species, such as hydroxyl radicals, that react with the methane molecules present.

5. The method of claim 1, wherein dry chemicals (e.g. dry urea hydrogen peroxide) containing hydrogen peroxide react with water vapor present to act as a source of molecular oxygen to create active oxygen species, such as hydroxyl radicals, that react with the methane molecules present.

6. The method of claim 1, wherein the metal cation catalysts that initiate the reaction are copper sulfate CuSO₄ or transition metal oxide cations such as PtO+, FeO+, and MnO+.

7. The apparatus of claim 2 wherein the reactor consists of a rotating mixing cell comparable in structure to that of a Wankel rotating engine.

8. The apparatus of claim 2 wherein the rotating mixing cell is designed to operate at negligible compression rate and only slightly elevated temperature.

9. The apparatus of claim 2 wherein the rotating mixing cell may be constructed of machined or cast metal, or low friction cast dielectric, non-metallic materials such as polypropylene or tetrafluoroethylene.

10. The apparatus of claim 2 wherein the reactor cell may be physically rotated by a separate Wankel engine connected to a common axle drive shaft, with both the reactor cell and the Wankel engine using a common source of methane as feed gas for methanol conversion and as an energy source to rotate the reactor.

11. The apparatus of claim 3 wherein the fibrous conversion screen may be composed of suitable dielectric materials such as polyethylene, polypropylene, and tetrafluoroethylene.

12. The apparatus of claim 3 wherein the conversion screen fibers may be coated with a fixed porous matrix structure composed of one or more silicon dioxide based sol gel formulations.

13. The apparatus of claim 3 wherein the metal cation catalyst needed to initiate the reaction may be imbedded in the conversion screen porous sol gel matrix, or coated directly on the fibers' surfaces, or on the walls of the reaction chamber, or both.

14. The apparatus of claim 3 wherein a humectant such as biodiesel, glycerin, or propylene glycol may be embedded on the oxygen release screen or its sol gel coating to facilitate retention of water vapor needed for oxygen release.

15. The apparatus of claim 3, wherein oxygen release screens are configured as endless belts for continuous replenishment of dry chemicals in a continuous conversion process.

16. The apparatus of claim 3 wherein multiple conversion cartridges are arrayed in the closed loop apparatus, with each conversion cartridge containing one or more coated conversion screens.
